(19) United States
(12) Patent Application Publication
(10) Pub. No.: US 2025/0354193 A1
(43) Pub. Date: Nov. 20, 2025

(54) COMPOSITION FOR REMOVING AND COLLECTING MICROORGANISMS

(71) Applicant: REALCO, Louvain-la-neuve (BE)

(72) Inventors: Laurent DELHALLE, Esneux (BE); Sébastien FASTREZ, Nouvelles (BE)

(73) Assignee: REALCO, Louvain-la-neuve (BE)

(21) Appl. No.: 18/874,050

(22) PCT Filed: Jun. 20, 2023
(86) PCT No.: PCT/EP2023/066578

§ 371 Date: Dec. 11, 2024

(30) Foreign Application Priority Data
Jun. 20, 2022 (BE) BE2022/5480
Jun. 20, 2022 (BE) BE2022/5481

(51) Int. Cl. C12Q 1/22; C12Q 1/04

(52) U.S. Cl. CPC C12Q 1/22; C12Q 1/04

(57) ABSTRACT

A sterile sampling composition includes a surfactant, one or more enzymes chosen from the group consisting of a protease, a polysaccharidase, a laccase, a lipase, a cellulase and a mannanase. A sampling kit includes the sterile sampling composition. A sampling method is based on the sterile sampling composition or kit and use of this composition or kit for the detection of bacteria on a surface.

### TECHNICAL FIELD

This invention relates to a composition for removing and collecting microorganisms, to its use, to a kit for removing, collecting and preserving microorganisms comprising said composition and to a method for removing, collecting and preserving microorganisms.

### PRIOR ART

In many fields of activity such as the agri-food sector, communities, medical and veterinary sectors, problematic contamination due to the presence of microorganisms is very frequently observed.

Traditionally, in hospitals and in medical or veterinary practices, the presence of microorganisms is responsible for nosocomial diseases while in the agri-food sector and in communities, these microorganisms are responsible for the degradation of perishable goods but also for the transfer of contaminants to consumers of products from, for example, a meat, fruit, vegetable or other production chain.

However, these days, strict hygiene standards are required and it is therefore necessary to ensure that the number of microorganisms responsible for such contamination is kept below an acceptable threshold value and this acceptable threshold value is specific to each field of activity.

Planktonic bacteria are a first example of contaminating microorganisms, these bacteria are free at the level of a liquid or solid substrate and pose a problem per se because they are able to directly contaminate any type of surface such as foodstuffs, medical tools, conveyor belts, storage tanks or even human patients or animals themselves.

However, today, it is widely recognised that the problems related to contamination by microorganisms are all the more pressing as they form biofilms.

Indeed, in any type of industry and more particularly in the field of the agri-food industry, biofilms are inevitably formed, given the richness of the surrounding environment.

Biofilms are defined as a consortium of microorganisms embedded in a matrix of extracellular polymeric substances. In other words, biofilms are viscous films that develop on all surfaces, following the adhesion of microorganisms to these surfaces and the secretion by these of polymers covering them, facilitating their adhesion to the surface and therefore forming the extracellular matrix. Biofilms therefore constitute a protective layer around microorganisms that is very resistant to chemical and thermal attack, which makes eliminating them using conventional biocides very difficult and they are also a recurring and significant source of contamination of the surrounding environment.

This resistance is explained on the one hand by the matrix of extracellular polymeric substances that forms a physical barrier to the diffusion of biocide molecules in the biofilm and, on the other hand, by the sessile state of microorganisms and their ability to very rapidly exchange genes responsible for certain biocide resistance mechanisms.

It is therefore easy to understand that biofilms are very resistant and extremely varied structures, for example different strains of the same bacteria can form different biofilms, and these biofilms are even more varied as they are composed of several different bacteria. In addition, bacteria exchange resistance genes between them and the environment also influences the biofilm, further increasing their complexity and resistance.

In addition, the extracellular matrix of biofilms can be identified when it is highly developed but, in the majority of cases, the biofilm develops insidiously in the facilities and its presence (or the presence of problematic microorganisms) will only be detected when the quality of the finished product is analysed.

With regard to the growth of the biofilm, this takes place in a cyclical manner, comprising a growth phase during which the accumulation of microorganisms occurs and a detachment phase during which whole pieces of biofilms and microorganisms detach themselves by erosion and under the effect of their weight to contaminate surfaces, foodstuffs, medical tools, conveyor belts, storage tanks or even human patients, consumers or animals themselves.

This leads to the manufacturer having to stop their production line and carry out a cleaning cycle to eliminate the biofilm, involving many hours of work and resources deployed and a loss of yield.

From the foregoing, it is shown that contaminating microorganisms and/or biofilms are a real issue, particularly in the field of healthcare (hospitals, dental or medical practices), veterinary care and the agri-food industry. This problem is all the more critical since microorganisms and/or biofilms can involve bacteria responsible for potentially fatal infections in individuals, whether these bacteria are present in hospitals, veterinary practices or in the agri-food industry and are ultimately found on food products intended for consumption.

As indicated above, the issue of microorganisms and more particularly biofilms is twofold. On the one hand, conventional disinfectants and biocides are very often ineffective because they fail to reach the microorganisms protected by the extracellular matrix of the biofilm, with this matrix having a complex and highly variable structure and composition. On the other hand, a biofilm is generally mixed, i.e. it contains a multitude of different bacteria or the same bacteria but which are of different strains, which promotes the spread of resistance genes between the bacteria of the biofilm and therefore makes the treatment thereof very difficult or even ineffective.

Consequently, from one environment to another or from one sector of activity to another, it is rather common for the biofilms detected to differ completely. In order to detect biofilms, there are kits for detecting the presence of biofilms on surfaces or in more specific facilities (water circuits, etc.); these kits (such as the one disclosed in document EP2537601) allow the selective staining of biofilms to be performed. It is therefore currently possible to determine areas where biofilms are present in order to eliminate them without knowing the precise nature of the microorganisms (bacteria) causing the target biofilm.

As a result, biofilm removal compositions comprising several enzymes are used without knowing whether the enzymes used and optionally formulated in a detergent base (see for example document EP2243821) are actually suitable to act on a given biofilm. For this reason, current treatments are rather random and non-specific, which is economically unprofitable and can lead to wasted time and a lengthy downtime of facilities.

Furthermore, techniques for collecting microorganisms from surfaces have been developed in order to characterise and/or quantify the populations of microorganisms present on surfaces and responsible for contamination. These collection methods therefore make it possible to determine, on a surface, the different types (strains) of bacteria and microorganisms present.

Among the methods for collecting microorganisms found on a surface, one of the references in this area is the ISO 18593:2004(F) Standard, which provides and defines horizontal methods for collection techniques on surfaces (more specifically on surfaces found in the context of the agri-food industry). The “cloth/sponge” method is an example of collection techniques cited in this Standard, with this method making it possible to search for and/or count microorganisms present on a surface. Briefly, this collection method involves wetting the cloth/sponge with a quantity of diluent which is sterile physiological serum, sampling the surface in two perpendicular directions before introducing the cloth/sponge into a sterile container with the diluent. Subsequently, after optional storage of the sample, the latter is analysed quantitatively and/or qualitatively.

Unfortunately, though such a method ensures the collection of free microorganisms on the surface of a substrate, i.e. the collection of planktonic microorganisms, it turns out to be ineffective when the surface is contaminated by microorganisms that have formed a biofilm. Indeed, as explained above, the microorganisms protected by the extracellular matrix of the biofilm, with this matrix having a complex and highly variable structure and composition and with the biofilms being mixed, are even more varied and complex as they are composed of several different bacteria or different strains.

In addition, it turns out that collection techniques based on a water-impregnated medium can have a beneficial effect on the development of microorganisms, even when performing a collection from a surface. Indeed, it is recognised that certain bacteria exhibit increased growth in the presence of water, the water-impregnated medium therefore promoting this growth during collection but also after said collection since a thin film of water remains on the treated media where collection took place. This can therefore promote the development of certain microorganisms that are not collected, in particular when they are protected by a biofilm.

In order to overcome the disadvantages of water-impregnated collection media, document DE10304331 is known from the prior art, which discloses an enzymatic preparation that can be used to eliminate biofilms from surfaces in the absence of biocides. This enzymatic preparation comprises one or more enzymes from the group of polysaccharidases and/or proteases and optionally nucleases.

Unfortunately, even though the compositions according to document DE10304331 have a certain effectiveness in terms of the collection of microorganisms, it appears that they are insufficient given the diversity and complexity of microorganisms and biofilms that may be present on a surface of a facility and that they do not allow for a representative collection of the bacterial populations that are nevertheless present there, i.e. they only allow for a low coverage of microorganisms.

Indeed, the biofilms and/or microorganisms present on a surface of a facility are on the one hand composed of a varied and complex population of microorganisms, i.e. different microorganisms but these different microorganisms may also still be of different strains, thus leading to different resistances but also the formation of varied, complex and different extracellular matrices of biofilms, sometimes within the same surface of a facility.

There is therefore an identified need to provide a composition that aims to address the disadvantages of the prior art by allowing effective, representative and adequate collection of microorganism populations, whether they are free or in the form of a biofilm with a complex structure that can vary considerably from one collection to another. In addition, there is a need to provide a composition allowing the collection of microorganisms on different types of surfaces, in different environments, which is stable over time and easy to use by users or compatible with the production chain, for example food in an agri-food industry.

In this respect, patent application WO 2018/002013 describes a viscous aqueous solution comprising one or more enzymes and 8.4% surfactants for application to a horizontal or even vertical surface, with a view to a prolonged impregnation of a tissue placed on this surface, so as to recover a maximum of microorganisms optionally present, including in the form of a biofilm, while preserving their viability and quantifying their presence.

In another technical field, patent EP0578666 discloses a composition for dishwashers comprising surfactants, enzymes and preservatives. The composition may, in one embodiment, be in liquid and concentrated form.

However, this publication does not describe polyol-type stabilising agents and provides information on the use of substances that are not compatible with sampling.

Similarly, patent U.S. Pat. No. 5,571,504 discloses a composition for treating contact lenses comprising a significant amount (1.75%) of detergents. Furthermore, again, the presence of polyol-type stabilising agents is not disclosed.

Patent application US2019/256803 describes a composition for destroying biofilms. This document does not describe the use of polyol-type stabilisers, or even sequestering agents.

Finally, patent JP5466782 describes a cleaning composition with anti-biofilm effectiveness and comprising a protease, propylene glycol and surfactants.

However, this document does not describe a sampling composition: the surfactant contents, or even their compatibility with sampling, are, for example, not specified.

### BRIEF SUMMARY OF THE INVENTION

This invention relates to a (sterile) composition for sampling microorganisms present on a surface, said sampling being compatible with downstream microbiological tests, comprising: one or more surfactants, present at a content of at least 0.01% by weight and at most 1.5% by weight relative to the weight of said composition (sum of the weights of surfactants: weight of the composition), said surfactant(s) comprising (or consisting essentially of) an anionic surfactant; at least two enzymes chosen from the group consisting of a (serine) protease, a polysaccharidase, a laccase, a lipase, a cellulase and a mannanase; at least one stabilising agent present at a content of at least 15% relative to the weight of said composition, said stabiliser being chosen from the group of polyols (preferably glycerol) and, preferably at least one sequestering agent.

This invention also relates to the use of this composition for collection for the detection of microorganisms, advantageously Salmonella sp. and/or Listeria sp.

### DETAILED DESCRIPTION OF AN EMBODIMENT OF THE INVENTION

The techniques for evaluating the presence of microorganisms include culturing, biochemistry (the measurement of ATP or NADH production, the measurement of enzymatic activity, for example catalase, or even the measurement of proteins in a medium), the genetic methods of “Polymerase Chain Reaction” (PCR) and sequencing (e.g. 16S ribosomal RNA) and immunological methods. In other words, many enzymes are used for biochemical analysis or for molecular biology analyses, flow cytometry or immunoassays, such as alkaline phosphatase, Taq polymerase, luciferase, etc. However, the inventors have noted that residual proteolytic activity interferes with these tests. In addition, proteases interfere with antigen-antibody complexes during immunological reactions, also widely used for the detection of microorganisms. Often several methods are combined, in particular the detection of pathogenic microorganisms potentially present at low intensity includes a culturing step, followed by immunological and/or genetic labelling. This means that the collection of microorganisms must ensure sufficient recovery, sufficient viability, but also that the products used do not interfere with downstream microbiological tests. There is therefore a very delicate balance to be respected.

To solve this problem, the invention provides a sterile composition for the sampling, removal and/or collection of microorganisms potentially present on a surface comprising:
at least one anionic surfactant, the surfactant(s) together representing at least 0.01% (preferably at least 0.02%) by weight relative to the weight of said composition (weight of surfactants: weight of the solution), and at most 1.5%, preferably at most 1%, or even 0.5%, or even 0.1%;
at least one polyol (glycerol) as a stabilising agent and/or viscosity agent representing at least 15%, preferably at least 18%, preferentially approximately 20% by weight relative to the weight of said composition, and/or preferably less than 30%, or even less than 25%, by weight relative to the weight of said composition,
advantageously, at least one sequestering agent (di- or multivalent cations), preferably at a content by weight of at least 0.2%, preferably at least 0.3%, or even at least 0.4%;
at least two enzymes (in a concentration capable of degrading any bacterial biofilms or stains and in a concentration not interfering with downstream microbiological tests), said at least two enzymes being chosen from the group consisting of a (serine) protease, a polysaccharidase, a laccase, a lipase, a cellulase and a mannanase.
Preferably, the surfactant(s) present in the collection composition (e.g. at a content of between 0.02% and 1.5%, or maximum 1%, maximum 0.5%, maximum 0.1%) consist (essentially) of one or more anionic surfactants.

This composition preferably has (i) a pH of between 5 and 11, preferably between 6 and 10, even more preferably between 7 and 9, or between 7.5 and 8 and/or (ii) a dynamic viscosity (20° C.) of between 1, preferably 2, 5, 10, 20, 30, 40, 50 mPa·s and 150, preferably 100, 90, 80, or even 70 mPa·s.

In the context of this invention, the word “surface” is to be understood broadly and preferably means a surface of objects or premises of private individuals, manufacturers, or even a public space (including public transport or public toilets) potentially contaminated by microorganisms, including pathogenic bacteria or microorganisms. For example, many surfaces in the agri-food industry risk being contaminated by potentially harmful microorganisms (toxic or spoilage), which is unacceptable and requires, where appropriate, the removal of infected batches. Examples of these surfaces may be tables, utensils, handles. Pharmacies, pharmaceutical industries, hospitals and medical practices also have many surfaces potentially contaminated by potentially harmful microorganisms, which must be identified quickly and precisely, which is what this invention allows.

Examples of microorganisms whose rapid and/or precise identification is beneficial, include, for the food industry, pathogenic microorganisms (for example, Escherichia coli, Salmonella sp., Listeria monocytogenes, Staphylococcus aureus, Bacillus cereus, Pseudomonas aeruginosa, Campylobacter jejuni, Clostridium perfringens, Aspergillus flavus), spoilage microorganisms (for example, Brochotrix thermosphacta, Lactaobcillus rahmnosus, Lactococcus piscium, Leuconostoc carnosum/gelidum, Paenibacillus sp, Zygosaccharomyces bailii/rouxi, Candida sp), or ferments (Lactobacillus casei, Lactococcus lactis, Lactobacillus sakei, Streptococcus thermophilus, Pediococcus acidilactici, Staphylococcus xylosus, Saccharomyces cerevisiae). More generally, the microorganisms potentially present on (non-food) surfaces and of interest to be identified quickly and/or precisely can be, for example Escherichia coli, Salmonella sp., Pseudomonns sp (e.g. P. aeruginosa), Staphylococcus sp. (e.g. S. aureus), Candida albicans, Alicyclobacillus sp., Stenotrophomonas sp., Acinetobacter sp., Klebsiella sp., Serratia sp., Bacillus sp., Ralstonia picketi and Burkholderia cepacia.

Unlike conventional cleaning or collection solutions, the detergents of this invention are advantageously chosen so as not to affect the viability of the bacteria to be collected: both their content and their chemical structure are important. Therefore, their concentration is less than 1.5% (weight of all detergents: weight of the entire composition). However, a minimum content, for example at least 0.01 or at least 0.02 or even 0.03% is necessary.

An example of a preferred detergent is laureth sulphate (or other “mild” anionic detergents, ideally a hydrocarbon chain of 12 to 14 carbon atoms, a poly(oxyethane-1,2-diyl) segment and an anionic head, for example a sodium sulphate).

The person skilled in the art knows other anionic detergents that are “mild”, or is even able to test other anionic detergents that are considered “mild”, for example by means of an in vitro test on microorganism culture (see below).

Preferably, the HLB index of all the emulsifiers in the composition is between 10 and 16.

In the context of this invention, the terminology HLB index (for “Hydrophilic-lipophilic balance”) relates to the hydrophilic or lipophilic character of the added surfactants.

Advantageously, the composition has been sterilised by physical means, for example by irradiation, such as gamma rays.

An advantageous alternative consists of sterilising the composition by sterilising filtration (e.g. passage through a filter with meshes of a diameter of 0.22 μm maximum) and/or by UVc irradiation (approximately 200 to 240 nm). Therefore, the sterilising filtration of the composition, followed by its incorporation into a bottle (or other container), followed by irradiation of this bottle (the container and its contents) with UVc is preferred because it ensures good sterilisation of the contents, and also of the container, which is important, for example for use in sensitive areas (hospitals, certain food industries, certain laboratories, etc.). In addition, the inventors have noted that the stability of the enzymatic composition is not significantly affected by this type of irradiation.

An alternative to sterilising filtration (or in synergy with it) is the formulation from quasi-sterile components. For example, the formulation from water comprising less than 10⁶, preferably less than 10⁵, preferably less than 10⁴, preferably less than 10³, preferably less than 10², preferably less than 10 CFU (colony forming units) per litre is preferred.

This formulation made from this quasi-sterile water, or the bottles comprising it, can then advantageously undergo sterilising irradiation, as described above (gamma, low intensity, electron beam, Uvc).

Preferably, this composition does not comprise a preservative. Alternatively, any preservative that may nevertheless be present must be as diluted as possible, or neutralised quickly (before applying the composition to a surface or just after), so as not to interfere with the metabolism of the microorganisms collected. Therefore, the inventors have noted that compositions, where the usual preservatives, including phenoxyethanol, were present at less than 50 ppm, were acceptable. The preferred compositions advantageously have a lower preservative concentration, for example less than 20 ppm, less than 10 ppm, less than 5 ppm (part per million: weight of the sum of the preservatives (e.g. phenoxyethanol): total weight of the composition), or even a concentration that is below the detection limit and/or 0.1 ppm.

Therefore, the choice of constituents (e.g. enzymes) forming the composition is partly based on their content of preservatives, the lowest possible and/or non-interfering preservatives. Therefore, a low residual content of 2-phenoxyethanol is acceptable, although it is better to keep it as low as possible.

This sampling composition is preferably for application on a surface, such as a surface used in the food industry. The composition can advantageously be applied to other industrial surfaces such as for example the pharmaceutical industry, the healthcare environment or even petrochemicals.

The characteristics of the composition according to this invention have the advantage of providing a composition for collecting microorganisms, whether they are in planktonic form or in the form of complex and varied biofilms. This composition allows the precise sampling of microorganisms.

The composition according to this invention, comprising at least two enzymes chosen from the group comprising a protease, a polysaccharidase, a laccase, a lipase, a cellulase and a mannanase, advantageously makes it possible to effectively degrade and deconstruct the extracellular matrix of biofilms and in particular a varied and complex population of biofilms having different extracellular matrices, therefore making it possible to release bacteria so they can be collected. However, the inventors have noted that the use of enzymes is particularly tricky when the microorganisms to be collected are sensitive and in small quantities.

Therefore, the viability of certain bacteria, in particular Gram-positive bacteria, is affected by several components: downstream tests that involve the culture or metabolism of such a bacterium could give false negative results, which is unacceptable (a “false positive” is less serious than a “false negative”, since the latter can be fatal). Indeed, Listeria is a feared contaminant, and the inventors have noticed that many components used prevented it from being detected downstream, which is all the more detrimental since this is accompanied by a good recovery of other microorganisms, which suggests that the method could have worked well. In addition to Listeria, the inventors have noticed that other Gram-positive bacteria were very sensitive to the different components of the collection solutions usually used.

Preferably, the composition comprises at least one protease, such as a serine protease (e.g. A subtilisin), the at least other enzyme being chosen from a polysaccharidase, a laccase, a lipase, a cellulase and a mannanase.

The use of a protease greatly increases the collection capacity of the composition but, as stated above, the inventors have noticed that this enzyme interferes with downstream tests and even, in certain cases, with other components of the collection composition itself.

Of course, protease activity can easily be inhibited, at least under laboratory conditions. However, the inventors have noticed that, in practice (on site, outside the laboratory), the available protease inhibitors were either complicated to use, ineffective, or interfered with downstream microbiological tests. Therefore, after many unsuccessful tests, the inventors prefer the incorporation of low-intensity protease activity. In the case of Alcalase (e.g. Alcalase 2.5 DX), this is preferably about 0.5 to 5 Anson Units per kg of the composition (0.2 g to 2 g of Alcalase 2.5 DX/Kg of composition), for example about 1 Anson Unit per kilo of the composition. Other proteases, including other commercial batches of Alcalase, may be used, for example serine proteases. The activity to be incorporated either corresponds to that mentioned above for alcalase (2.5 DX) or is determined in light of the examples of this invention: the highest concentration that does not interfere with downstream microbiological tests.

The person skilled in the art is well aware of how to test for proteolytic activity, for example by using a substrate whose colouration (or fluorescence properties) changes after proteolytic cleavage. Therefore, the act of determining activity equivalent to that of a certain amount of alcalase 2.5 DX is within their knowledge. Alternatively, or in addition, preferably, the substrate used is N-succinyl-alanyl-alanyl-propyl -phenylalanyl-p-nitroanilide (CAS 70967-97-4), which is dissolved in a phosphate buffer at pH 7.25 mM at a concentration of 1 mM. 1 ml of this substrate brought to 20° C. is placed in the presence of 100 μl of the solution to be tested (also at 20° C.), for example at pH 7, or at an optimal pH for enzymatic activity and absorbance is measured based on time. The enzymatic activity is then easily determined downstream, for example by the Beer-Lambert law. In other words, in light of the above, the act of determining an equivalent activity is a routine activity. For example, an activity equivalent to 0.2; 0.3; 0.4; 0.5; 0.6; 0.7; 0.8; 0.9; 1; 1.5 or 2 g/kg of alcalase 2.5 DX advantageously means, in the context of this invention, that the protease considered (e.g. a serine protease, or even another subtilisin) has the same activity under the conditions described above (substrate CAS no. 70967-97-4).

In light of this invention, a routine test can be developed, either as an alternative or in combination, by mixing several dilutions of a Listeria culture with the above low amount of alcalase, or with a saline solution or with another given protease (at several concentrations), followed by measurement by a specific Listeria detection test: the saline composition and the alcalase (applied at a low dose) representing two positive controls.

In the non-preferred case where a protease inhibitor is added after collection, the protease concentrations can be revised upwards. However, the nature and concentration of the protease inhibitor must, in a particularly preferred manner, be determined so as not to interfere with downstream microbiological tests.

In addition, the sequestering agent and the pH between 5 and 11 (between 7 and 8.5) of the composition according to this invention advantageously make it possible to form complexes with mineral ions which will be fixed in a form that prevents the precipitation thereof by the usual reactions and therefore to stabilise the composition according to this invention over time, in particular in view of the enzymes present in such proportions.

In the context of this invention, the sequestering agent is preferably a chemical substance having the capacity to form complexes with mineral ions which it fixes in a form that prevents the precipitation thereof by the usual reactions. For example, the sequestering agent may be glucono-delta-lactone, sodium gluconate, potassium gluconate, calcium gluconate, citric acid, phosphoric acid, tartaric acid, sodium acetate, sorbitol, a compound comprising a phosphorus atom, or even carboxymethyl inulin (the sodium salt). So-called “mild” sequestering agents are preferred: the inventors have noted that these “mild” sequestering agents allow for better enzymatic activity at the collection level and/or for downstream microbiological tests involving enzymes (e.g. the inventors have noted interference with downstream PCR tests when too many sequestering agents and/or overly strong sequestering agents are present). Alternatively, the sequestering agent may be a phosphorus oxide such as a phosphonate, a phosphinate or a phosphate or a mixture thereof, or a salt thereof, an amine or an amine oxide carrying at least, in its structure, a phosphine, phosphine oxide, phophinite, phosphonite, phosphite, phosphonate, phosphinate or phosphate functional group, alone or in combination, or a salt thereof. Carboxymethyl inulin is advantageous and the inventors have shown that this compound does not interfere with downstream microbiological tests. Similarly, mixtures of carboxymethyl inulin with a glycolate (e.g. in a mass ratio of between 0.5:1 and 2:1; carboxymethyl inulin: sodium glycolate) are preferred. The inventors have also obtained good results with phosphonates.

In the context of this invention, it has been determined that the composition according to the invention, comprising at least one anionic surfactant (comprising one or more surfactants, said surfactant(s) being (essentially) one or more anionic surfactants) which represents at least 0.01% by weight relative to the weight of this composition (preferably at least 0.02%, and less than 1.5%, preferably less than 1%, or even less than 0.5%, or even less than 0.1%), makes it possible in a particularly advantageous manner to remove and collect a representative and complete proportion of a population of microorganisms present on a surface, whether they are planktonic or in the form of biofilms since the enzymes of the composition according to the invention allow the release thereof. There is therefore a fine balance between the nature of the detergents, their low concentration, the nature of the enzymes and low activity (protease).

Advantageously, this anionic surfactant consists of a foaming surfactant or a foaming surfactant is incorporated into the anionic surfactant(s), which has the advantage on the one hand of removing and collecting the microorganisms and on the other hand of trapping the microorganisms collected in the composition according to the invention in order to carry out an effective and representative collection of the population of contaminants from a surface.

In addition, the composition according to this invention comprising at least one anionic surfactant and at least one polyol or propylene glycol as a stabilising agent and/or viscosity agent at least 15% by weight, is stable over time.

Furthermore, it has been determined that the composition according to this invention, by not being too viscous, makes it possible to significantly reduce the contact time required with the microorganisms and/or biofilms present on a surface so as to deconstruct them sufficiently in order to release the bacteria and therefore be able to sample them and/or identify them for the specific and appropriate subsequent treatment of the surfaces. In other words, the composition according to this invention therefore makes it possible to completely surround the microorganisms or biofilms and to deconstruct them more quickly and more efficiently, therefore providing an adequate and optimal collection composition, and also making it possible to trap the microorganisms and contaminants collected from a surface without encountering the issue of rapid evaporation of the liquid collection compositions.

As a result, the composition according to this invention, unlike current compositions, makes it possible to obtain a truly representative and sufficiently comprehensive collection of the contaminants present on a surface, for different types of surfaces and for different environments. This is particularly advantageous because by having a representative and complete sample of microorganisms, they can subsequently be identified and a targeted and effective elimination treatment, using the appropriate compounds and no longer a cocktail of compounds which is often ineffective, can be applied to the surface. This is of course a considerable advantage in terms of economics and time.

The benefit of the composition according to this invention is therefore twofold in that it makes it possible, on the one hand, to collect and sample all the contaminants from a surface, whether they are planktonic or in the form of biofilms, for the purpose of analysing them, and that it then makes it possible to determine precisely which treatment will be applied to eliminate these contaminants.

Preferably, as described above, the at least one anionic surfactant of the composition according to the invention is chosen from the group comprising sodium laureth sulphate. This molecule has the CAS number 9004-82-4. This is preferably sodium laureth-2 sulphate and/or sodium laureth-3 sulphate

Preferably, the at least one polyol or fatty alcohol as a stabilising agent and/or viscosity agent is glycerol. The inventors have noted that glycerol (or other polyols), when present in large quantities (for example more than 15% by weight), increases the stability of the composition over time.

Preferably, the composition according to the invention further comprises a pH regulating agent.

Preferably, the composition according to the invention has a pH of between 6 and 10, preferably between 7 and 9, preferentially between 8 and 9, particularly advantageously between 8.2 and 8.6.

Preferably, the composition according to the invention is in liquid form, preferably in sprayable liquid form.

Preferably, the composition according to the invention further comprises at least one additional enzyme chosen from the group consisting of oxidoreductases, lyases, transferases, lipases, esterases, glucosaminidases.

As explained above, the benefit of the composition according to this invention is twofold in that it makes it possible, on the one hand, to collect and sample all the contaminants from a surface whether they are planktonic or in the form of biofilms and that it then makes it possible to precisely determine the nature of these contaminants, which makes it possible to know which treatment will be applied to eliminate these contaminants.

Consequently, it is important that the composition according to the invention, which contains the contaminants, microorganisms and biofilms following collection, can allow for a reliable, correct and representative analysis of the population collected without said composition after collection interfering with subsequent analyses.

The invention therefore also relates to a kit for sampling, removing, collecting and preserving microorganisms (in a living or revivable state) for subsequent laboratory analyses.

This kit comprises the composition described above, and a means of controlled application thereof. For example, a sprayer calibrated so as to project the same quantity of composition, with the same dispersion and in droplets of identical volume.

In other words, this invention provides a kit for sampling, removing (from a surface), collecting and preserving microorganisms in a living or revivable state for subsequent laboratory analyses comprising:
a composition for sampling (removing and collecting) microorganisms according to this invention,
at least one device for the controlled application of this sampling composition (removing and collecting) on the surface;
preferably at least one means for the sterile harvesting of these microorganisms,
preferably at least one sterile container, for example a sterile bag, comprising a solution for preserving microorganisms for subsequent laboratory analyses,
preferably, this kit further comprises means for specific detection and/or specific quantification by immunology or genetics of bacteria, preferably chosen from Salmonella (e.g. Salmonella enterica, Salmonella bongori), Pseudomonas (e.g. Pseudomonas aeruginosa) and/or Listeria (e.g. Listeria monocytogenes).

Preferably, the at least one means for sterile harvesting of the microorganisms of the kit according to this invention is a tissue, a wipe or a sponge.

The preservative solution, in the context of this invention, preferably comprises polyoxyethylene sorbitan a and/or phosphoglyceride derivative (e.g. lecithin); this composition preferably has the role of neutralising any potential biocidal activity that could be present in the sampling composition, for example caused by the presence of preservatives that would always be present or of disinfecting agents that are accidentally present.

Preferably, the preservative composition comprises a phosphate buffer (e.g. pH 7, 25 mM) which may advantageously further comprise salts, such as magnesium chloride and/or calcium chloride.

This preservative solution may advantageously further comprise a protein hydrolysate (e.g. peptone) or a protein-rich compound, so as to provide nutrients to the collected microorganisms and/or to counteract the negative effects associated with residual protease activity, if present.

This preservative solution advantageously comprises reducing thiol groups, for example by cysteines (in reduced form) and/or glutathione. Thiols that are not of this nature are also possible, provided that they are compatible with the metabolism of the microorganisms. When the thiol group is cysteine, it may be present by the protein hydrolysate, for example a protein hydrolysate rich in cysteine and/or where reducing potential has been ensured.

Preferably the polyoxyethylene sorbitan derivative of this kit is polyoxyethylene sorbitan monooleate and/or the phosphoglyceride derivative is phosphatidylcholine.

Advantageously, the polyoxyethylene sorbitan derivative, when present, is present at a content of at least 4 g/L, preferably at a content of at least 4.3 g/L, preferentially at a content of at least 4.6 g/L, advantageously at a content of at least 4.9 g/L. Similarly, the phosphoglyceride derivative, when present, is present at a content of between 0.5 and 3 g/L, preferably at a content of between 0.5 and 2.5 g/L, preferentially between 0.5 and 2 g/L, advantageously between 0.5 and 1.5 g/L

Preferably, the protein hydrolysate (peptone) is present at a content of at least 10 g/L, preferably at least 11 g/L, preferably at least 12 g/L, preferentially at least 13 g/L, advantageously at least 14 g/L, particularly advantageously at least 15 g/L.

The invention further relates to a method for sampling, removing, collecting and preserving microorganisms in a living or revivable state for subsequent laboratory analyses comprising the following steps:
a) at least one controlled application of a composition for sampling, removing and collecting microorganisms according to this invention,
b) allowing this sampling (removal) composition to act on this surface to be sampled,
c) depositing at least one means for harvesting these microorganisms on this surface to be sampled,
d) leaving this harvesting means to be soaked by this sampling (removal) composition,
e) performing a sterile collection of this soaked microorganism harvesting means and depositing it in at least one sterile container, for example a sterile bag comprising a solution for preserving microorganisms in a living or revivable state for subsequent laboratory analyses.

Advantageously, step b) of the method according to the invention takes place for a duration of at least 10 seconds, at least 20 seconds, at least 30 seconds, at least 1 minute, preferably at least 2 minutes, preferentially at least 3 minutes, advantageously at least 4 minutes, particularly advantageously at least 5 minutes and, preferably, less than 30 minutes, preferably less than 15 or 10 minutes.

Preferably, the method according to the invention further comprises at least one preliminary step of preheating this sampling (removal) composition, preferably at a temperature of between 20 and 60° C., preferentially between 30 and 50° C., advantageously between 40 and 50° C.

Preferably, the method according to the invention further comprises at least one step of storing this sterile container containing this soaked harvesting means, at a temperature below 15° C., preferably below 10° C., advantageously below 5° C., such as from 4 to 8° C. Alternatively, storage can be carried out at less than 0° C., provided that the microbiological activity can be subsequently restored.

Preferably, the controlled application of a volume of the sampling composition is an application of a content of between 0.5 and 2 grams of composition (per 100 cm²), preferably of a content of between 0.7 and 1.8 grams of composition per application, preferentially between 0.9 and 1.6 grams of composition (per 100 cm²), advantageously between 1.1 and 1.4 grams of composition (per 100 cm²).

Preferably, the sterile harvesting means is a tissue, wipe, swap [sic], scraper, swab or sponge.

Advantageously, the subsequent laboratory analyses of step e) described above comprise one or more of the following tests:

a test involving a step of culturing microorganisms;

a biochemical test (e.g. ATP-metry, NADH detection, catalase activity measurement);

a test comprising an immunological recognition step, and

a molecular biology test (a test step involving markers and/or probes and/or genetic primers and a combination of these steps, sequencing).

As will be described below, these tests are skewed by collection solutions from the prior art, but are now possible when the collection solutions of this invention are used, even for the most sensitive microorganisms, such as Listeria sp., or microorganisms present in low quantities (CFU).

Therefore, another related aspect of this invention relates to the use of the composition or kit described above for the detection of contamination of the genus Salmonella on surfaces, preferably of the genus Salmonella enterica and/or Salmonella bongori and/or for the detection of contamination of the genus Listeria on surfaces, preferably of the genus Listeria monocytogenes and/or for the detection of contamination of the genus Pseudomonas on surfaces, preferably of the genus Pseudomonas aeruginosa and/or for the detection of contamination of the genus Staphylococcus on surfaces, preferably of the genus Staphylococcus aureus.

In other words, this aspect of this invention relates to the use of a liquid collection composition comprising one or more surfactants and one or more (e.g. at least 2) enzymes chosen from the group consisting of a protease, a polysaccharidase, a laccase, a lipase, a cellulase and a mannanase, for the detection of bacteria chosen from Salmonella (e.g. Salmonella enterica, Salmonella bongori), Pseudomonas (e.g. Pseudomonas aeruginosa) and/or Listeria (e.g. Listeria monocytogenes) potentially present on a surface.

Preferably, following this use, detection is carried out by immunology or by genetic testing and/or after culturing the bacteria.

Preferably, in this use, the collection composition comprises surfactants present at a content of between 0.02% and 1% (or even 1.5%; preferably between 0.1% and 0.5%) by weight relative to the weight of said collection composition.

Preferably, in this use, the surfactant(s) is (are) anionic, advantageously chosen from the group consisting of alkyl ether sulphate, preferably a salt of laureth sulphate, alkyl sulphate, alkylbenzene sulfonate and soaps, preferably the surfactants are essentially one or more anionic surfactants.

Preferably, in this use, the collection composition further comprises at least 15% of a stabilising agent chosen from a polyol, preferably glycerol, and propylene glycol, relative to the weight of said collection composition, and/or at least one sequestering agent (di- or multivalent cations).

Preferably, in this use, the collection composition comprises a protease which is a serine protease

Other characteristics, details and advantages of the invention will be discussed in the description given below, in a non-limiting manner and with reference to the examples.

### EXAMPLES

The first obstacles that the inventors had to overcome relate to the lack of representativeness of the biofilms generated in the laboratory, compared with the situation on site. This specifically complicates the choice of enzymatic activities (nature of enzymes, their abundance, presence of potentially harmful compounds). Based on these preliminary tests, the inventors therefore decided that all compositions that would be formulated in the laboratory should be tested in real conditions: many formulations that seem promising in the laboratory cannot be used, in practice, and the inventors have not found published guidelines, even with the help of their expertise, that could have helped them to identify functional compositions. However, once a complex formulation has been identified, such as those described below, some compounds in this formulation can be replaced by others, which can be easily tested in the laboratory (effectiveness, absence of toxicity for microorganisms), then on site, or directly on site. Therefore, to a certain extent, one protease can be substituted by another, in particular when these are serine proteases (subtilisins). However, when commercial enzymatic compositions are used, the inventors have noticed that, depending on the suppliers, and even the brands, different additives may interfere with the microbiology analyses, therefore variations in the composition of this invention are possible, thanks to the teaching of this invention, but the rules of caution described in this invention must be followed.

### Example 1

A meat processing company located in Belgium was chosen for this study. All the facilities were previously cleaned and disinfected before performing the collections.

The removal protocol is applied with limited mechanical actions that could introduce a reproducibility error; it includes the following steps:

Quickly scan (screen) the areas to undergo collection with the UV lamp and BDK detection kit (Realco);
Preheat the collection solution to 45° C.;
Place two templates on the surfaces to undergo collection (in order to define a surface of 10 cm by 10 cm): physiological water vs. collection composition (14 sprays to cover the area; 1.2 g/spray);
Leave the collection solution to act for 5 minutes;
Place the sterile wipe on the two collection areas and leave it to soak;
Perform sterile collection using forceps and place in a sterile transport bag;
Store the samples in refrigerated containers at 4° C.;
Then evaluate the two areas with the lamp and the BDK.

A total of 6 collection areas were included in this study: Zone 1 round table; Zone 2 lid for rubbish table; Zone 3 stainless steel bin; Zone 4 saw table; Zone 5 plastic surface; Zone 6 derinding machine.

In these 6 zones, the collection composition comprising enzymes (including a subtilisin, activity equivalent to 10 Anson Units/kg) and detergents (approximately 10% by weight) allows a much better recovery of microorganisms, both in classical microbiology and by ATP measurement (2G) or by culturing. Conversely, there were no more microorganisms on the surfaces where the collection solution was placed, unlike the surface treated with physiological water. The difference is especially obvious at the level of classical microbiology, compared with ATP 2G or by testing involving a culturing step (little difference noted).

The inventors reproduced the example, to include metagenomic analyses, while varying the contact time of the collection composition on the surface (30 seconds and 5 minutes). Again, the enzymatic composition allows for much better collection (e.g. 100 times better in classical microbiology and 14 times better in qPCR). The bacteria found show an increased proportion of Pseudomonas.

### Example 2—Addition of a Preservative Solution; Salmonella

The inventors, suspecting harmful effects associated with the collection composition, sought to determine to what extent an adjuvant solution could counteract these, in the context of tests aimed at determining the presence of Salmonella and/or Listeria, either by PCR or qPCR, or by using commercial detection kits, for example those from BioMérieux. To do this, a so-called ‘preservative’ solution was tested (in g/l): Proteins (beef extract; 5); Peptone, 10; Polysorbate 80 (Tween 80; 5); Lecithin 0.7; NaCl, 5. In addition, various serine protease inhibitors were used, in order to neutralise the subtilisin activity of the collection solution.

The first series of tests was performed in the laboratory, where known quantities of bacteria (0.1 ml of 10⁷ CFU/ml Salmonella) were incubated either in physiological solution (100 ml), or in the collection composition of Example 1 (6 ml+94 ml physiological water), or in the composition of Example 1, before the preservative solution was added (6 ml collection composition, 90 ml water, 4 ml preservative solution).

When 10⁷ UFC/ml of Salmonella are analysed (i.e. 10⁴ CFU/ml in the final mixture), the different situations are compatible with the identification of the microorganism by means of PCR analyses or the Vidas® Salmonella test. However, detection by the Vidas SLM® test (culture broth, then specific measurement by antigenic test and fluorescence; this test provides information on the absence or presence of Salmonella) fails when the collection composition is present, whereas it works when the bacteria are diluted in physiological water or when the preservative solution has been added.

Furthermore, in this example, the inventors consider that the Salmonella concentrations are high, which does not necessarily, or even rarely, represent the situation in the field, where even 1 CFU must be detected.

### Example 3—Addition of a Preservative Solution; Listeria

In this case, the inventors noticed that the enzymatic solution causes problems at the detection level (enumeration and qPCR); these problems are reduced thanks to the preservative solution, however the Vidas® commercial kit (LMX) still does not work.

Furthermore, when the amount of Listeria inoculated is reduced, the microorganism is generally no longer detected following mixing (Vidas LMX®, never; Vidas LMO2®, not every time) with the collection composition, while detection remains strong when mixing was carried out in physiological water only. In this case (which is intentionally extreme), the preservative composition did not improve detection. As a precaution, the tests were reproduced and three strains of Listeria were tested with the same results every time; these strains were ATCC 19114, NCTC 11994 and ATCC 13932.

By comparing all the results, the inventors conclude that the collection composition slows the growth of microorganisms (Listeria), a problem that is partially solved by the preservative composition. On the other hand, the collection composition interferes with certain downstream microbiological tests. Since the nature and abundance of microorganisms on a surface to be tested is unknown in advance, the inventors therefore conclude that the compositions of Examples 1 to 3 are useful, but would benefit from being further improved.

### Example 4—Identifying Harmful Compounds

The inventors investigated various commercial enzymes to see if any compounds interfered with downstream microbiological tests. The inventors noticed that the presence of compounds such as phenoxyethanol, potassium sorbate or thiazolinone derivatives is quite widespread. Thus, the inventors selected enzymatic compositions which are free from such compounds, or insofar as possible, even though the inventors found significant concentrations of 2-phenoxyethanol in many commercial enzymatic compositions. The compounds tested included Bronopol, triclosan, idocarb, chlorhexidine, DCOIT, BIT, OIT, CIT, MIT, ethylparaben, methylparaben, butylparaben, phenylparaben, isopropylparaben, isobutylparaben, propylparaben, salisylic acid, 4-hydroxybenzoic acid, 4-methylbenzoic acid, benzoic acid, sorbic acid, dehydroacetic acid, 1-phenoxypropan-2-ol, 1,2-dibromo-2,4-dicyanobutane, 2-hydroxybiphenyl, 2-phenoxyethanol (found in highest quantities, sometimes up to 0.4% by weight), bensyl alcohol and chlorophenesin.

### Comparative Example 1

Suspecting interference between the proteases in the collection composition and the enzymes or antibodies in the downstream tests, the inventors tested the effect of adding protease inhibitors to the preservative solution (see example 3), while avoiding commercial enzymatic compositions comprising compounds identified as harmful (see example 4) as much as possible.

Different ways of applying the protease inhibitor were tested. Since the chosen protease was a subtilisin or trypsin, two serine protease inhibitors were tested: PMSF and AEBSF.

PMSF allowed good inhibition of the proteolytic activities of the serine proteases tested. On the other hand, AEBSF only inhibited trypsin. Measurement tests where Listeria (the 3 strains above were tested) is brought into contact with a composition comprising a subtilisin, then AEBSF in a concentration sufficient to cause complete inhibition of subtilisin have shown that different microbiological tests gave lower values, or did not even detect Listeria under the test conditions, unlike the control without the enzymes or when PMSF is added.

Therefore, the inventors chose to incorporate PMSF into the preservative solution to be tested, despite the disadvantages of this molecule.

Unfortunately, even under these conditions, the Vidas® LMX test does identify the presence of Listeria monocytogenes.

### Example 5—Optimisation of the Collection Composition

The inventors then decided to test the effectiveness of a highly diluted collection composition avoiding the compounds identified as harmful as much as possible. In addition, enzymatic activities are reduced, as is the detergent content. Detergents are chosen after laboratory tests based on their compatibility with the life of microorganisms, including Listeria, which has been identified as more sensitive.

First of all, with detergent concentrations of 10% by weight, even in the presence of low enzymatic activity, in the presence of minimal preservative content (e.g. almost completely non-detectable; phenoxyethanol less than 4 ppm) and in the absence of PMSF, Listeria detection tests, such as Vidas®, do not work well (significant underquantification), or do not work in a reproducible manner. Even less specific tests do not work when a Listeria culture is subjected to these different treatments.

Therefore, all the parameters of the composition must be checked, with the problem that an overly high dilution of enzymatic activities risks no longer allowing the removal of microorganisms, while an overly high dilution of detergents risks no longer allowing the even application of the composition or good contact with the biofilm. Therefore, the inventors noticed that, in addition to the quantity of the components, the nature of the components had to be reconsidered.

A diluted formulation was tested comprising about 20% by weight of glycerol, about 0.03% by weight of an anionic detergent of the laureth sulphate type (sodium), 0.4% by weight of sequestrant (carboxymethyl inulin); 0.4% by weight of sodium glycolate; 0.04% by weight of a subtilisin (activity 1 Anson Unit), as well as a lipase; an amylase, a cellulase and a mannanase. The inventors selected enzymes, here commercial enzymes, without preservatives such as potassium sorbate, MIT, OIT, BIT and CIT. When possible, the inventors used commercial enzymes free from phenoxyethanol (cellulase and mannanase), or with a low content of this molecule (subtilisin; 4 g/kg of the enzymatic formulation, lipase; 0.249 g/kg and amylase; 0.233 g/kg). Enzymes other than subtilisin were added in weight contents of between 0.1 and 0.5% (weight of the enzymatic composition: total weight) and the amount of enzyme is typically 1 to 10% by weight relative to the commercial formulation.

Following the use of this composition, the presence of Listeria is detected, and the Vidas® LMX test for Listeria is, ultimately, positive and reproducible. The inventors further noted that, under these diluted conditions, the addition of the protease inhibitor PMSF is not necessary, and even reduces the spread of Listeria.

The inventors consider that the concentration of enzymes other than proteases (here, a subtilisin) can still be somewhat reduced, e.g. the lowest concentration that allows detachment, as well as those of the sequestering agent. In the light of this invention, this can be tested in the laboratory on the 3 strains of Listeria or in other models, including, preferably, more complex ones.

The inventors then tested a possible synergy with the preservative solution (see examples 2 and 3): since the results are already very good, the improvement associated with the addition of the preservative solution is marginal, but it was nevertheless interesting in the case of a test that includes spreading followed by detection by PCR.

It is understood that this invention is in no way limited to the embodiments described above and that many modifications can be made thereto without departing from the scope of the appended claims.

1. A sterile composition for sampling microorganisms potentially present on a surface comprising:
one or more surfactants, present at a content of at least 0.02% by weight and at most 1.5% by weight relative to a weight of said composition, said one or more surfactants comprising an anionic surfactant;
at least two enzymes chosen from the group consisting of a protease, a polysaccharidase, a laccase, a lipase, a cellulase and a mannanase;
at least one stabilising agent, preferably present at a content of at least 15% relative to the weight of said composition, said stabiliser being chosen from the group of polyols,
at least one sequestering agent.

2. The sampling composition according to claim 1, comprising at least one anionic surfactant chosen from the group consisting of alkyl ether sulphate, a salt of laureth sulphate, alkyl sulphate, alkylbenzene sulfonate and soaps.

3. The sampling composition according to claim 1, wherein the sequestering agent is chosen from glucono-delta-lactone, sodium gluconate, potassium gluconate, calcium gluconate, citric acid, phosphoric acid, tartaric acid, sodium acetate, sorbitol, carboxymethyl inulin and mixtures thereof.

4. The sampling composition according to claim 1 comprising at least 50% (by weight) water.

5. The sampling composition according to claim 1, wherein the stabilising agent is glycerol.

6. The sampling composition according to claim 1 comprising at least one protease, in a concentration equivalent to 0.5 to 5 Anson Units of Alcalase 2.5 DX per kg of said composition.

7. (canceled)

8. The sampling composition according to claim 1, comprising a sprayable liquid.

9. The sampling composition according to claim 1 comprising less than 100 ppm of 2-phenoxyethanol (part per million: weight of 2-phenoxyethanol: weight of the composition).

10. The sampling composition according to claim 1 comprising from 0.02 to 0.5% of surfactant(s), said surfactant(s) consisting essentially of one or more anionic surfactants.

11. A kit for sampling microorganisms potentially present on surfaces, in a living or revivable state, for subsequent detection and laboratory analysis comprising:
a sampling composition for sampling microorganisms from a surface, according to claim 1,
at least one device for controlled application of said sampling composition to said surface,
means for specific detection and/or specific quantification by immunology or genetics of bacteria chosen from Salmonella, Pseudomonas and/or Listeria.

12. The sampling kit according to claim 11 further comprising at least one sterile container, and/or means for sterile harvesting of microorganisms.

13-25. (canceled)

26. The sampling kit according to claim 11 further comprising at least one sterile bag, and/or a tissue, a wipe or a sponge for sterile harvesting of microorganisms.
